(19) United States
(12) Patent Application Publication
(10) Pub. No.: US 2023/0310939 A1
(43) Pub. Date: Oct. 5, 2023

(54) FITNESS SYSTEM

(71) Applicant: Shapelog, Inc., Ann Arbor, MI (US)

(72) Inventors: Nolan Orfield, Ann Arbor, MI (US); Bryan O'Rear, Wilmette, IL (US); Mark Angeloni, Chicago, IL (US)

(73) Assignee: Shapelog, Inc., Ann Arbor, MI (US)

(21) Appl. No.: 18/295,058

(22) Filed: Apr. 3, 2023

Related U.S. Application Data
(60) Provisional application No. 63/362,341, filed on Apr. 1, 2022.

(51) Int. Cl. A63B 24/00; A63B 71/06

(52) U.S. Cl. CPC A63B 24/0075; A63B 24/0006; A63B 24/0062; A63B 71/0619; A63B 2024/0009; A63B 2024/0068; A63B 2220/40; A63B 2220/803; A63B 2220/833; A63B 2220/836

(57) ABSTRACT

A fitness system includes a computing system in communication with a wearable device and multiple pieces of exercise equipment. The wearable device is associated with a user, includes a user sensor for detecting motion, and outputs user sensor data. The multiple pieces of exercise equipment are configured to be used by the user to perform an exercise therewith, include an equipment sensor for detecting motion, and output equipment sensor data. The computing system receives and processes the user sensor data collected at a first time and the equipment sensor data collected from the multiple pieces of exercise equipment at the first time to associate the user with one of the multiple pieces of exercise equipment used by the user at the first time, determines exercise information, exercise recommendations, or both, and transmits the exercise information, the exercise recommendations, or both.

### CROSS-REFERENCE TO RELATED APPLICATION(S)

This application claims priority to and the benefit of U.S. Provisional Patent Application No. 63/362,341, filed Apr. 1, 2022, the entire disclosure of which is incorporated by reference.

### BACKGROUND

Fitness enthusiasts seek ways to track their workout progress and improve performance, such as strength or fitness. In the context of weight training, a person may record the exercise performed, the amount of weight used, the number of repetitions performed, the rate of perceived exertion, etc. For cardio exercise, a person may track how long they exercised, the average speed, the average distance, resistance settings, and so on. Simple ways to track workout progress include documenting the workout in a logbook. Apps have also been developed that allow users to document their workouts using, e.g., their mobile device. It would be advantageous to provide systems that simplify tracking workouts, provide coaching, or both.

### FIELD

The present disclosure related to exercise equipment and, in particular, systems for tracking exercise information.

### SUMMARY

An fitness system includes a computing system in communication with a wearable device and multiple pieces of exercise equipment. The wearable device is associated with a user, includes a user sensor for detecting motion of the user, and outputs user sensor data according to the detecting of the motion of the user. Each one of the multiple pieces of exercise equipment is configured to be used by the user to perform an exercise therewith, includes an equipment sensor for detecting motion of the piece of the exercise equipment, and outputs equipment sensor data according to the detecting of the motion of the one piece exercise equipment. The computing system receives the user sensor data from the wearable device and the equipment sensor data from the multiple pieces exercise equipment. The computing system processes the user sensor data collected at a first time and the equipment sensor data collected from the multiple pieces of exercise equipment at the first time to associate the user with one of the multiple pieces of exercise equipment used by the user at the first time, determines exercise information, exercise recommendations, or both from the equipment sensor data collected from the one piece of exercise equipment at the first time, and transmits the exercise information, the exercise recommendations, or both.

### BRIEF DESCRIPTION OF THE DRAWINGS

FIG. 1 illustrates an example fitness system.

FIG. 2 is a block diagram illustrating example components of a wearable device used in the fitness system of FIG. 1.

FIG. 3 is a block diagram illustrating example components of exercise equipment used in the fitness system of FIG. 1

FIG. 4 is a block diagram illustrating example components of a local server used in the fitness system of FIG. 1.

FIG. 5 is a block diagram illustrating example components of a remote server used in the fitness system of FIG. 1.

FIG. 6 is a flowchart of an example process that may be executed by the fitness system of FIG. 1.

FIG. 7 is a flowchart of an example process that may be executed by the wearable device of FIGS. 1 and 2.

FIG. 8 is a flowchart of an example process that may be executed by the local server of FIGS. 1 and 3.

FIG. 9 is a flowchart of an example process that may be executed by the remote server of FIGS. 1 and 4.

FIG. 10 is a schematic diagram illustrating an example automatic activity detection and data pull performed by the fitness system of FIG. 1.

### DETAILED DESCRIPTION

Fitness tracking apps are popular among gym-goers. The biggest downside to existing fitness tracking apps, however, is that they require manual input of workout information. The user may have forgotten how many reps they performed, misjudged their rate of perceived exertion (RPE), distance, forgotten how much weight was used during the exercise, forgotten the number of sets they performed, etc.

One solution involves automatically tracking and recording a user's exercise activity. Even that proves challenging, however. First, some users object to automatic tracking for privacy reasons. Moreover, not all fitness equipment is equipped with the appropriate sensors and electronics for accurately tracking exercises and associating such exercises with a particular user.

A fitness tracking system that can track and record a user's workout while respecting the user's privacy and without requiring special gym equipment is disclosed. The fitness tracking system includes a wearable device, a local server, and a remote server. The remote server includes a memory and a processor programmed to execute instructions stored in the memory. The instructions include receiving real-time data captured by the wearable device and generating an exercise recommendation based at least in part on the real-time data captured by the wearable device.

The elements shown may take many different forms and include multiple and/or alternate components and facilities. The example components illustrated are not intended to be limiting. Indeed, additional or alternative components and/or implementations may be used. Further, the elements shown are not necessarily drawn to scale unless explicitly stated as such.

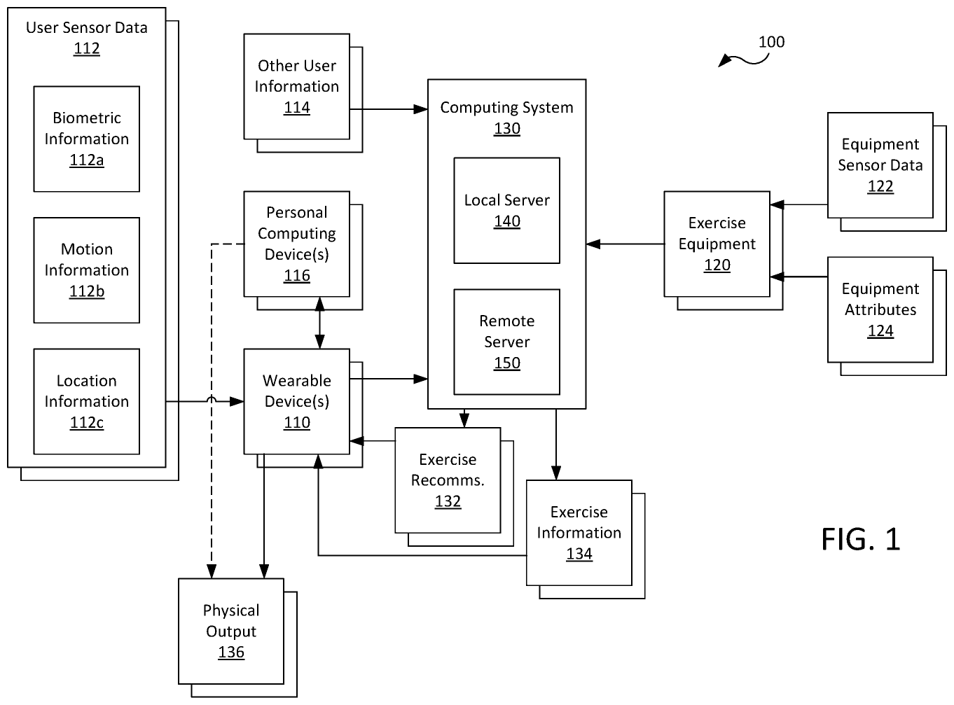

As illustrated in FIG. 1, a fitness system 110 includes at least one wearable device 110, exercise equipment 120, and a computing system 130. The at least one wearable device 110 may include multiple of the wearable devices 110, each associated with a different one of multiple users. The exercise equipment 120 may include multiple pieces of the exercise equipment 120, such as different exercise machines, such as weight or resistance machines and cardio machines, or other exercise equipment, such a dumbbells or free weights. The term exercise equipment 120 may refer singularly to one piece of the exercise equipment or plurally to multiple pieces of exercise equipment. The exercise equipment 120 may also be referred to as an exercise machine, exercise machines, piece of exercise equipment, or pieces of exercise equipment. The computing system 130 may include a singular computing device (e.g., a server or other type of computer), or may include multiple computing devices (e.g., a local server 140, and a remote server 150). In order to distinguish between different instances of common terms (e.g., users, pieces of exercise equipment, time, data, information, etc.), different identifiers may be used (e.g., “one user” vs. “another user” and “the other user,” “first time” vs. “second time,” “the user sensor data” vs. the “other user sensor data,” etc.).

The wearable device 110 is implemented via circuits, chips, or other electronic components that can collect real-time data 112 about a user through various sensors located on the wearable device 110. The real-time data 112 pertaining to the user (e.g., the wearable device 110) may also be referred to as real-time user data or user sensor data 112. The wearable device 110 may be further configured to provide information to the user and may still further be configured to provide physical outputs 118 that are detectable by the user. The physical outputs 118 may be provided in one or more forms of visual, audible, or tactile outputs, or a combination thereof. The physical outputs 118 may, for example, include physical alerts, physical instructions, or both. The physical alerts output to the user may indicate a condition to a user, such as the completion of an exercise, point within an exercise, or availability of information from the wearable device 110. The physical instructions output to the user direct the user to perform an action, for example, in accordance with a recommendation for the exercise currently being performed by the user. The instructions may, for example, pertain to timing, effort, or equipment settings as discussed in further detail below.

The user sensor data 112 may include biometric information 112a such as the user's heart rate. Additionally or in the alternative, user sensor data 112 may include motion information 112b that identifies user movements, hand positions, etc. In some possible implementations, the user sensor data 112 may include location information 112c which may reflect a relative or absolute location of the user. The relative location may be the location or distance of the user relative to an object with a known location (e.g., a location relative to a particular piece of exercise equipment or other devices). The absolute location may be the location of the user relative to a coordinate system such as the Global Positioning System (GPS). The wearable device 110 is further configured to wirelessly communicate with the computing system 130, such as the remote server 150 and/or the local server 140, and possibly other electronic devices, such as a personal computing device 116 associated with the user of the wearable device 110 (e.g., a smartphone, tablet computer, laptop computer, or desktop computer). The biometric information 112a may also be referred to as user biometric information or user biometric sensor data. The motion information 112b may also be referred to as user motion information or user motion sensor data. The location information 112c may also be referred to as user location information or user location sensor data. The user sensor data 112 is discussed in further detail below.

The terms “real-time data,” “real-time information,” or similar generally refer to types of data or information that may vary and are collected at a then-current time (e.g., the motion information 112b of acceleration and/or rotational velocity at successive times). Any such real-time data or information may be used (e.g., processed) at later times after collection and still be referred to as real-time data or information.

Each piece of the exercise equipment 120 is implemented via circuits, chips, or other electronic components that can collect real-time data 122 about a piece of the exercise equipment 120 through various sensors of the exercise equipment 120. The equipment sensor data 122 may, for example, include information about movement and/or a setting of the exercise equipment 120 and/or information derived therefrom. The real-time data 122 pertaining to the exercise equipment 120 may be referred to as real-time equipment data, activity data, exercise data, or equipment sensor data 122.

As discussed in further detail below, the user sensor data 112 pertaining to the user and the equipment sensor data 122 pertaining to the exercise equipment 120 may be cooperatively processed, alone or in conjunction with other data, in order identify the different users of different pieces of the exercise equipment 120 at different times and/or to later provide information collected from the exercise equipment 120 to the user (e.g., exercise recommendations 132 and/or exercise information 134).

The computing system 130 is implemented by one or more computing devices (e.g., servers) itself implemented via circuits, chips, or other electronic components, for example, as described for the local server 140 and/or the remote server 150. The computing system 130 may be configured and programmed to directly or indirectly receive, store, process, generate, and/or transmit information about the user (e.g., the user sensor data 112 and/or other user information 114 from the wearable device 110 and/or other sources), about the exercise equipment 120 (e.g., the equipment sensor data 122 and/or equipment attributes 124 from the exercise equipment 120 and/or other sources), and/or combinations thereof (e.g., exercise recommendations 132 and/or exercise information 134). While specific functions and operations are discussed below as being performed specifically by each of the local server 140 and the remote server 150, those same functions may instead be performed by the computing system 130 and/or may be performed by the computing system 130 having a singular computing device and/or additional computing devices, while removing and/or adding appropriate operations for transmitting data therebetween.

As described below, the other user information 114 may include a user profile, user demographics, historical exercise information associated with the user (e.g., previous number of repetitions performed of an exercise, a previous amount of weight lifted, a previous number of sets completed, etc.), target exercise information (e.g., a target number of repetitions the user wishes to perform a target amount of weight the user wishes to lift, a target number of sets the user wishes to complete, etc.), user attributes such as the user's sex, gender, age, height, or weight, the user's training goals, etc. The other user information 114 may also include regular location information, for example, identifying a particular facility that the user may frequent. The regular location information may be used to filter (e.g., prevent) associations with the exercise equipment 120 at other locations.

In some implementations, the other user information 114 stored or otherwise sent to the computing system 130 (e.g., the local server 140, the remote server 150, or both) does not include personal identifiable information (PII) that would otherwise permit the computing system 130 to identify an individual to whom the information applies to be reasonably inferred by either direct or indirect means. The user sensor data 112 and/or the other user information 114 may be may be anonymized when sent to and/or utilized by the computing system 130 for associating the user sensor data 112 to the equipment sensor data 122, generating and transmitting the exercise recommendations 132 and/or the exercise information 134, or both, for example, by excluding any personal identifiable information (e.g., name, address, or set of demographic information from which the user may be identified). The user information 114 sent to, stored by, or both the computing system 130 includes suitable information from which the exercise recommendations 132 can be generated, for example, relating to the historical exercise information, target exercise information, training goals, and/or appropriate biometric information that may influence the exercise recommendations (e.g., age, weight, height or ranges thereof).

In one example, the user sensor data 112 and any of the other user information 114 are sent to and stored by the computing system 130 in association with an anonymized user identifier. The anonymized user identifier may, for example, be a unique number or set of alphanumeric characters that is fixed or regenerated at appropriate events (e.g., each day, each time entering a particular exercise facility, each time starting a new exercise). The anonymized user identifier allows the association of the user sensor data 112, the other user information 114, and the equipment sensor data 122, generating the recommendations 132 for the user, and transmitting the recommendations 132 and the exercise information 134 to the user (e.g., the wearable device 110 of the user).

As also described below, the equipment attributes 124 may represent the type of exercise equipment, body part(s) exercised when using the machine or apparatus, weight increments or resistance settings of the machine or apparatus, the location of the machine or apparatus in a gym, etc. The equipment attributes 124 may also include a facility identification and/or location, which may identify a particular exercise facility at which the exercise equipment 120 is located and/or a specific location therein. The equipment attributes 124 may also include other pre-determined characteristics about the exercise equipment 120 or the exercises conducted thereon, including mechanical characteristics thereof (e.g., path of motion of user and/or equipment, orientation of the equipment or users through the path of motion or different stages thereof), which may be fixed or may change for different settings of a machine (e.g., being reconfigurable). The equipment attributes 124 may generally correspond to the user sensor data 112 of the user collected by the wearable device 110 (e.g., user wrist motion corresponding to motion of a handle of the exercise equipment 120) or information derived therefrom (e.g., type of exercise).

As described in further detail below, the exercise recommendations 132 include information about one or more exercises currently being performed or to be performed by the user at a future time. For exercises currently being performed on a piece of the exercise equipment 120, the exercise recommendations 132 may include, for example, maintaining output, changing current output (e.g., increasing or decreasing cadence or difficulty), and/or changing length (e.g., duration of cardio exercise or number of repetitions within a set). For exercises to be performed in the future, the recommendations 132 may, for example, include increasing an amount of weight lifted, increasing a time under tension, performing more repetitions, performing more sets, exercising auxiliary muscles, exercising for longer periods of time, exercising for shorter periods of time, stretching, etc. As also described in further detail below, the exercise information 134 may represent an exercise performed by the user with the exercise equipment 120, for example, summarizing the exercise performed (e.g., naming or otherwise identifying the exercise and quantifying the exercise, such as by repetitions, time, weight, work, and/or other measure).

In one example, the local server 140 is implemented via circuits, chips, or other electronic components configured and programmed to collect information about the user and their workout, process the information, and deliver information about the user and their workout to the remote server 150. The information delivered to the remote server 150 may include data stored in a user profile on the local server 140 (e.g., the other user information 114), the user sensor data 112 captured by the wearable device 110, the equipment sensor data 122 captured by the exercise equipment 120, and/or the equipment attributes 124. Moreover, as discussed in greater detail below, the local server 140 may receive the exercise recommendations 132 and the exercise information 134 about the exercises performed by the user from the remote server 150 and update the user profile with such recommendations and the exercise information 134. In some instances, the local server 140 is located at a gym or other exercise facility. Alternatively, the local server 140 may be a cloud-based server configured to communicate with the wearable device 110, the exercise equipment 120, and the remote server 150 via a network such as the internet.

In one example, the remote server 150 is implemented via circuits, chips, or other electronic components configured and programmed to communicate with the local server 140. For instance, the remote server 150 may be programmed to receive, from the local server 140, the user profile stored at the local server 140 (e.g., the other user information 114), as well as the user sensor data 112 collected by the wearable device 110 and/or the equipment sensor data 122 collected from the exercise equipment 120 and/or the equipment attributes 124. In some possible approaches, the remote server 150 is located at a gym or other exercise facility. In other possible implementations, the remote server 150 is a cloud-based server configured to communicate with the wearable device 110, the local server 140, or both, via a network such as the internet.

In one possible approach, the remote server 150 may receive the user sensor data 112 and other user information 114 from the local server 140. The other user information 114 may include a user profile, user demographics, historical exercise information associated with the user (e.g., previous number of repetitions performed, a previous amount of weight lifted, a previous number of sets completed, etc.), target exercise information (e.g., a target number of repetitions the user wishes to perform a target amount of weight the user wishes to lift, a target number of sets the user wishes to complete, etc.), user attributes such as the user's sex, gender, age, height, or weight, the user's training goals, etc. The user sensor data 112 such as the motion information 112b, biometric information 112a, and location information 112c may be collected by the wearable device 110 and transmitted to the remote server 150 by way of the local server 140. As discussed above, the local server 140 may transmit the other user information 114, such as the user profile, user demographics, historical exercise information, target exercise information, user attributes, etc., to the remote server 150.

The remote server 150 may be further configured or programmed to receive or access equipment attributes 124 associated with particular exercise equipment, such as a weight machine, cable machine, treadmill, rower, or the like. The equipment attributes 124 may represent the type of exercise equipment, body part(s) exercised when using the machine or apparatus, weight increments or resistance settings of the machine or apparatus, the location of the machine or apparatus in a gym, etc. The remote server 150 may be further configured or programmed to receive the equipment sensor data 122 (e.g., exercise data) captured during use of exercise equipment such as a weight machine, cable machine, barbell, dumbbell, etc. The equipment sensor data 122 captured by the exercise equipment 120 may represent an amount of weight lifted, a number of repetitions performed, a number of sets performed, an average repetition speed, a time under tension, and so on.

The remote server 150 may be configured or programmed to process the received user sensor data 112 of the user, the other user information 114, the equipment attributes 124, and equipment sensor data 122 from the exercise equipment 120 by comparing the user sensor data 112, the other user information 114, the equipment attributes 124, and the equipment sensor data 122 to data stored in various databases, tables, records, and other reference material. As such, the remote server 150 may be configured or programmed to identify an exercise performed by the user, generate exercise recommendations 132 for the user, and/or generate exercise information 134 representing the exercise performed by the user based, at least in part, on the user information and exercise data received and processed. In some instances, the exercise recommendations 132 and the exercise information 134 may be fed back into the remote server 150 to improve future exercise recommendations 132 and the exercise information 134 provided by the remote server 150. That is, the remote server 150 may apply machine learning to improve the accuracy of the exercise recommendations 132 over time. Moreover, the exercise recommendations 132 may be transmitted to the local server 140 so the user profile may be updated accordingly and/or communicated to the user through the wearable device 110 and/or the personal computing device 116 thereof, such as a mobile device such as the user's cell phone, or a computer such as a tablet computer, a laptop computer, or a desktop computer. For example, after receipt of the exercise recommendations 132 and the exercise information 134 from the remote server 150, the local server 140 may transmit the exercise recommendations 132 and the exercise information 134 to the wearable device for presentation to the user. Alternatively or in addition, the local server 140 may transmit the exercise recommendations 132 and the exercise information 134 to the personal computing device 116 (e.g., the user's mobile device) via, e.g., an app, may generate an email sent to the user, or may make the exercise recommendations 132 and the exercise information 134 available to the user via a website.

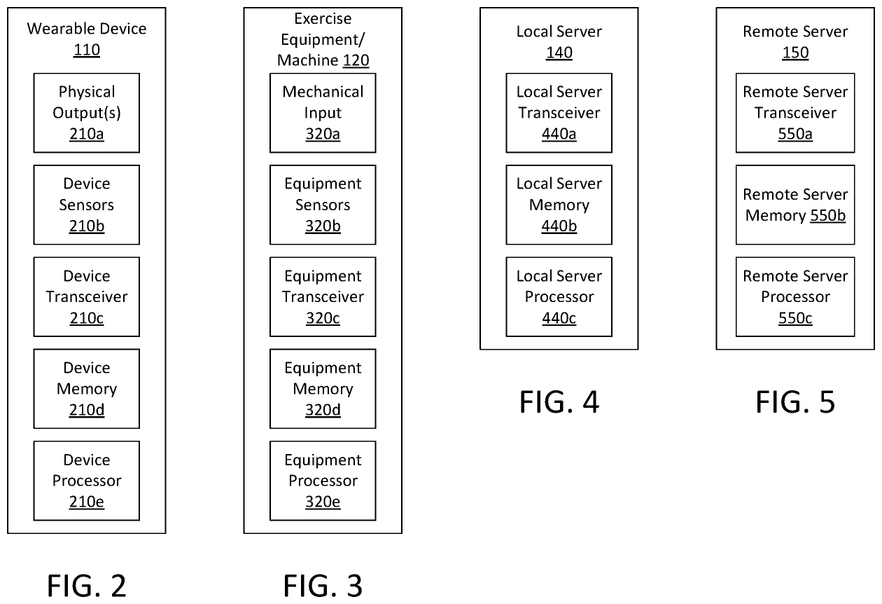

FIG. 2 is a block diagram illustrating example components of the wearable device 110. As shown in FIG. 2, the wearable device 110 includes one or more wearable device physical outputs 210a, wearable device sensors 210b, a wearable device transceiver 210c, a wearable device memory 210d, and a wearable device processor 210e.

The one or more wearable device physical outputs 210a may, for example, include a visual output, an audio output, and/or a tactile output device. The one or more wearable device physical outputs 210a are configured to provide an output to the user, such as an alert or instructions. An alert is generally an indicator to a user. Instructions are directives to users to perform certain actions. The visual output device may be configured as a display screen, such as an LCD, OLED, or other type of display, or a light indicator that provide to the user information, alerts, and/or instructions. In some instances, the wearable device display screen is a touchscreen. The wearable device display screen may be configured to present wearable device information to the user. The wearable device information may include user sensor data 112 captured by the wearable device sensors 210b, the biometric information 112a, the location information 112c, the exercise recommendations 132, the exercise information 134, the other user information 114, the user profile, or the like. The audio output device may include, for example, a speaker that outputs tones and/or verbal outputs to the user to provide alerts or instructions. The tactile output device may include, for example, a haptic output device (e.g., a vibratory output) that outputs vibrations in one or more patterns to the user to provide alerts and/or instructions. In some possible implementations, the wearable device physical outputs 210a include haptic output devices (e.g., haptic feedback sensors). The haptic output device may include various actuators or other sensors that vibrate or emit a sound for purposes of getting the attention of the user. For instance, the haptic output device may vibrate or output a sound in response to a signal from the wearable device processor 210e. The vibration or sound may represent the receipt of updated user information or a new exercise recommendation 132 or new exercise information 134, otherwise provide alerts or instructions to the user.

The wearable device 110 may be configured to provide the physical outputs 118 according to the exercise recommendation 132 or the exercise information 134 received thereby. In one example, the physical output 118 is an alert that provides the user awareness of a condition, which may pertain to exercises performed by the use or to operations of the fitness system 100. For example, the physical output 118 may include a visual alert (e.g., display of an image, illumination, or flashing), an audible alert (e.g., a tone or verbal output), or a tactile alert (e.g., a vibratory output of a predetermined pattern) to indicate a milestone within or completion of an exercise (e.g., at a time threshold or repetition threshold within an exercise, or completion thereof) or to indicate receipt of the exercise recommendation 132 or exercise information 134 by the wearable device 110 or the personal electronic device 116 of the user. In one instance, the physical output 118 may include a visual alert that displays the exercise recommendation 132 or the exercise information 134.

In another example, the physical output 118 is an instruction that directs the user to perform an action, for example, pertaining to the exercise being performed by the user. For example, the physical output 118 may be one of differentiated outputs that provide differentiated instructions to the user, for example, based on the exercise recommendation 132. The differentiated instructions may, for example, include instructions to two or more of maintain, reduce, or increase the cadence (e.g., speed) of an exercise currently being performed by the user (e.g., to change the speed of repetitions or running speed). The differentiated instructions may, for example, to change a setting of the exercise equipment 120 (e.g., resistance, configuration, speed, or inclination). The differentiated instructions may, for example, include instructions to stop or continue an exercise (e.g., to stop performing repetitions, to perform more repetitions, to stop a cardio exercise, or to continue a cardio exercise, which may be relative to a planned number or repetitions or time duration). The physical outputs 118 for each of the differentiated instructions may include differentiated visual outputs (e.g., different alphanumeric instructions or color coded instructions), audio outputs (e.g., different tones, sequence of tones, or number of tones), or tactile outputs (e.g., different vibration patterns, sequence of pulses, or number of pulses).

The wearable device sensors 210b are implemented via circuits, chips, or other electronic components that can detection user activity in real time. For instance, the wearable device sensors 210b may include one or more motion sensors, such as accelerometers, gyroscopes, or an inertial measurement unit (IMU). The wearable device sensors 210b may also be referred to as user sensors. The motion sensors are configured to output acceleration data measured by the one or more accelerometers of the IMU (e.g., in three axes) and/or angular velocity data measured by the one or more gyroscopes of the IMU (e.g., about three axes of rotation). In some instances, the wearable device sensors 210b include biometric sensors configured to capture biometric information 112a about the user. In that possible implementation, the wearable device sensors 210b may include a heart rate sensor. The wearable device sensors 210b may be configured or programmed to detect the real-time activity by the user and output signals representing the real-time activity of the user, such as during the performance of an exercise, to the wearable device processor 210e.

The wearable device sensors 210b may include other types of sensors that may be capable of detecting motion or other aspects of the user or their environment, such as the exercise equipment 120. For example, the wearable device sensors 210b may include visual sensors, such as a camera (e.g., visible light, infrared, or both), structured light, time of flight, or combinations thereof. The visual sensors may utilize suitable algorithms for detecting or classifying motion, objects or features, or both, such as machine vision algorithms. In one example, the visual sensor is configured to detect visual indicia that identifies the exercise equipment 120 used by the user, such as a quick response (QR) code, to associate the wearable device 120 with particular pieces of the exercise equipment 120 (as opposed to cooperatively processing motion data from the wearable device 110 and the exercise equipment 120 for association therebetween).

The wearable device transceiver 210c is implemented via an antenna, circuits, chips, or other electronic components that can transmit and receive signals from other devices including the local server 140, the remote server 150, or both. For instance, the wearable device transceiver 210c may be configured or programmed to transmit the user sensor data 112 to the local server 140, the remote server 150, or both. Additionally or in the alternative, the wearable device transceiver 210c may be configured or programmed to receive the other user information 114, the exercise recommendations 132, and the exercise information 134 from the computing system 130, such as the local server 140, the remote server 150, or both. The wearable device transceiver 210c may be configured or programmed to communicate in accordance with any number of wired or wireless communication protocols. For instance, the communication transceiver may be programmed to communicate in accordance with a satellite-communication protocol, a cellular-based communication protocol (5G, LTE, 3G, etc.), Bluetooth®, Bluetooth® Low Energy, Ethernet, WiFi, ANT, etc.

The wearable device memory 210d is implemented via circuits, chips, or other electronic components and can include one or more of read only memory (ROM), random access memory (RAM), flash memory, electrically programmable memory (EPROM), electrically programmable and erasable memory (EEPROM), embedded MultiMediaCard (eMMC), a hard drive, or any volatile or non-volatile media etc. The wearable device memory 210d may store instructions executable by the wearable device processor 210e and data such as the user sensor data 112, the other user information 114, the exercise recommendations 132, the exercise information 134, or the like. The instructions and data stored in the wearable device memory 210d may be accessible to the wearable device processor 210e and possibly other components of the wearable device 110, such as the wearable device transceiver 210c and/or the wearable device outputs 210a.

The wearable device processor 210e is implemented via circuits, chips, or other electronic components and may include one or more microcontrollers, one or more field programmable gate arrays (FPGAs), one or more application specific integrated circuits (ASICs), one or more digital signal processors (DSPs), one or more customer specific integrated circuits, etc. The wearable device processor 210e is configured or programmed to receive the output of the wearable device sensors 210b and generate, from the output of the wearable device sensors 210b, the user sensor data 112 representing activities of the user.

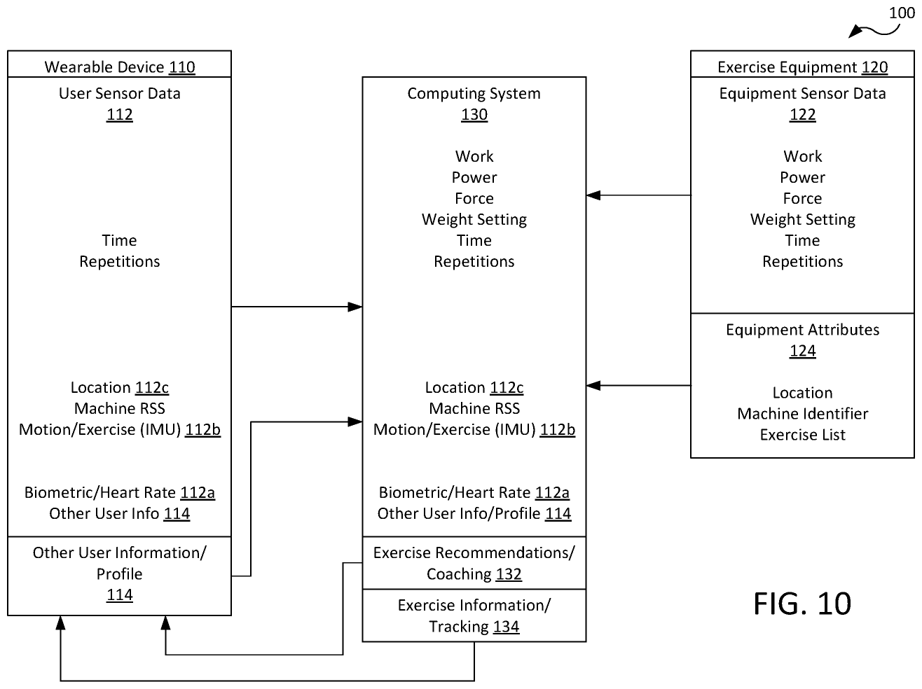

The user sensor data 112 may include the motion information 112b in raw form (e.g., the linear acceleration and/or angular velocity data received from the motion sensors) or as processed from the raw data by the wearable device processor 210e or the computing system 130. As processed from the raw data, the user sensor data 112 may quantify various characteristics of exercises performed by the user, which may be referred to as user exercise characteristics. The user sensor data 112, such as the raw data or the user exercise characteristics, may be later processed with the equipment sensor data 122, such as raw data or equipment exercise characteristics derived therefrom, to associate the user sensor data 112 and/or the wearable device 110 with the equipment sensor data 122 collected from the particular piece of the exercise equipment 120 being used by the user of the wearable device 110 at a given time. The user sensor data 112 and the other user information 114 may, for example, include those types of information illustrated in FIG. 10.

Examples of the user exercise characteristics and include, for example: repetition start, peak, and end positions and corresponding times; performance of a repetition and corresponding time; repetition distance and corresponding repetition number or time; repetition duration and corresponding repetition number or time; repetition motion; number of repetitions in a set; repetition start, peak, and ending orientation, and/or repetition orientation path; cadence; and/or exercise type. A repetition generally refers to a repeated motion of an exercise (e.g., lifting of weight during a resistance exercise, arm swing or step while running, stroke during rowing, etc.).

TABLE 1
User Exercise Characteristics

| User Sensor Information | Description; Source |
|---|---|
| User location | Detected exercise facility and/or location therein |
| Linear acceleration (1-3 axes) | Measured from wearable device sensors 210b (e.g., |
| | accelerometer or IMU) |
| Angular velocity (1-3 axes) | Measured from wearable device sensors 210b (e.g., gyroscope |
| | or IMU) |
| Repetition start position | Repeated position of user (e.g., wrist) at start of each repetition |
| Repetition peak position | Repeated position at the end of each repetition, generally |
| | furthest from the start position |
| Repetition end position | Repeated position at the end of each repetition, generally |
| | forming the start position of each successive repetition |
| Performance of a repetition | Binary determination of whether and when a repetition has |
| | been performed by the user |
| Repetition distance | Distance between start and peak position |
| Repetition motion | Varying position in space between start and peak positions |
| Repetition duration | Time span between start and end positions |
| Repetition cadence | Rate of repetitions |
| Number of repetitions | Number of times moving from starting to peak positions |
| Set duration | Time span between start of first repetition and end of last |
| | repetition |
| Repetition start orientation | Orientation of user (e.g., wrist) in start position |
| Repetition peak orientation | Orientation of user in peak position |
| Repetition end orientation | Orientation of user in end position |
| Repetition orientation path | Varying orientation between start and peak positions |
| Exercise | General classification or specific identification of exercise |
| | being performed |

The positions and orientations of the user (e.g., the wrist or other location of the wearable device 110) may be calculated in any suitable manner from the raw motion data from the accelerometer, gyroscope, and/or IMU (e.g., the linear acceleration and/or angular velocity data). For example, positional data may be derived from the linear acceleration data (e.g., double integrating) with repeated minimums and maximums being determined over time with any suitable method (e.g., tables, plots, function fitting, etc.). The minimums represent the start and end positions of each repetition, the maximums represent the peak positions of each repetition, and the distance therebetween represents the distance of reach repetition. The time between successive minimums represents the repetition duration, the number of maximums represents the number of repetitions, and the time between the first minimum and the last minimum represents the set duration. The start, peak, and end orientations of each repetition coincide in time to the start, peak, and end positions of each repetition. Cadences may be calculated as a rate of repeating the peak position. Exercise type may be determined according to the repetition motion, repetition orientation path, and/or other information. For example, during a repetition of resistance exercise or cardio exercise, the repetition may follow certain motions and/or orientations, as well as cadences (e.g., repetitions during arm curl exercises, arm swing while running, and/or stroke while rowing). The exercise type may be determined, for example, using a machine learning or other suitable algorithm (e.g., comparison or pattern recognition to those of known activities).

The wearable device processor 210e may be further configured or programmed to command the wearable device transceiver 210c to transmit the user sensor data 112, such as the raw data or the user exercise characteristics, to the local server 140. In some instances, the wearable device processor 210e may be programmed to access and process the other user information 114, the exercise recommendations 132, and the exercise information 134 from the local server 140 or the remote server 150 by way of the wearable device transceiver 210c.

The wearable device processor 210e may be further configured or programmed to command the physical outputs 210a to provide the physical output 118, as described above, for example in response to the exercise recommendation 132, the exercise information 134, other user sensor data 112, or combinations thereof, such as an alert, instruction, or a visual or graphical representation of the other user information 114, the exercise recommendations 132, the exercise information 134 via the wearable device physical output 210a. In some instances, the wearable device processor 210e is further configured to output a signal to the haptic feedback sensor upon receipt of updated user information, a new exercise recommendation 132, and/or new exercise information 134. As discussed above, the haptic feedback sensor may vibrate or emit a sound to alert the user of the wearable device 110 of the updated or new information.

FIG. 3 is a block diagram illustrating example components of one piece of the exercise equipment 120. As shown in FIG. 3, each piece of the exercise equipment 120 generally includes a mechanical input 320a, one or more exercise equipment sensors 320b, an exercise equipment transceiver 320c, an exercise equipment memory 320d, and an exercise equipment processor 320e.

The mechanical input 320a is moved during a user's performance of an exercise. In some implementations, the exercise equipment 120 is a resistance machine (e.g., a weight machine) in which case the mechanical input 320a receives and provides resistance to force input by the user in performance of one or more resistance exercises. The amount of resistance may be selectable by the user and be provided, for example, by weights, springs, or other mechanisms. In other implementations, the exercise equipment 120 is a cardio machine in which case the mechanical input 320a receives or causes repeated movement of the user during performance of a cardio exercise (e.g., a rowing machine receiving repeated strokes of the user, or a treadmill moving a belt requiring repeated steps of the user).

The mechanical input 320a may define exercise motions, which are paths of motion of the user through which the user applies the force in the case of resistance machines (e.g., wrist motion during different types of arm press, curl, or extension exercises) or paths of motion attendant to cardio exercises (e.g., wrist motion during rowing strokes, or wrist motion attendant to running).

The mechanical input 320a may define a singular fixed exercise motion, several selectable fixed exercises motions, a singular generalized exercise motion, or several selectable generalized exercise motions. In the case of fixed exercise motions, the mechanical input 320a may include one or more linkages by which force is transferred from the exercise equipment 120, such that the force is applied through the same motion (e.g., an arm curl machine with a fixed axis of rotation and handle position that is fixed or selectable relative thereto). In the case of generalized paths for resistance machines, the mechanical input 320a may include one or more flexible cables by which force is transferred from the user to the resistance, such that force is applied through same general but not exact motion (i.e., holding the cable in tension) (e.g., an arm press machine having arms that are fixed or selectively move the handles between bench, chest, and shoulder press positions). The different exercise motions for each selection and whether fixed or generalized have different characteristics (e.g., start, end, and peak positions, orientations; distance traveled; path traveled within space), which may be unique between different exercise equipment 120 and/or the exercise motions. The equipment attributes 124 include the exercise motions and/or the measurable characteristics thereof. For example, equipment attributes 124 (e.g., motion attributes) may include identification or other characterization of the exercise performed thereon, a repetition path within space and repetition orientation through the repetition path, as described in Table 2 below.

TABLE 2
Equipment Attributes

| Equipment/Motion Attribute | Description; Source |
|---|---|
| Exercise | General classification of exercise (e.g., chest, arms, legs, |
| | cardio), specific exercise (e.g., chest press or arm curl), or |
| | specific exercise on specific machine (e.g., chest press on |
| | particular machine type with particular configuration or |
| | setting) |
| Facility | Exercise facility and/or location therein |
| Repetition path in space | Path of motion through space defined by mechanical input |
| | 320a, may vary for different settings or configurations of the |
| | exercise equipment |
| Repetition orientation through | Varied orientation of user interface with the mechanical input |
| path | 320a through the repetition path, which may vary for different |
| | settings or configurations of the exercise equipment |

The equipment sensors 320b are configured to detect and collect the equipment sensor data 122 from the exercise equipment 120. The equipment sensor data 122 may include the performance and/or parameters of different exercises being performed by users with the exercise equipment 120, which may include settings of the exercise equipment 120 (e.g., resistance, weight, or speed settings; position, or exercise selections), and motion (e.g., movement of the mechanical input 320a, such as the linkage, cable, or resistance, including travel), as well as the times corresponding to the equipment sensor data 122 collected.

The equipment attributes 124 and the equipment sensor data 122 may, for example, as shown in FIG. 10, include work, power, force, weight setting, time, repetitions, location, machine identifiers, exercises, combinations thereof, and information derived therefrom. As referenced above and discussed in further detail below, the user sensor data 112 from the wearable device 110 may be processed with the equipment attributes 124 and/or the equipment sensor data 122 in order to identify particular users of particular exercise equipment 120 at a given time, for example, by identifying or associating the wearable device 110, the user sensor data 112 collected thereby, or both with the exercise equipment 120, the equipment sensor data 122 collected thereby, or both.

The equipment sensors 320b may include touch and/or force sensors (e.g., capacitive sensors, pressure sensors, and/or strain gauges) that detect application of force and/or the amount of force being applied by the user to the mechanical input 320a, motion sensors (e.g., magnetic and/or optical sensors) configured to detect motion of the mechanical input 320a. The equipment sensors 320b may further include configuration sensors (e.g., optical sensors and/or switches) that are configured to directly or indirectly detect parameters of the exercise being performed by the user, such as the amount of resistance and/or configuration of the exercise equipment 120 (e.g., different exercise motions that may be indicative of the different exercises to be performed therewith by the user).

The equipment transceiver 320c is implemented via an antenna, circuits, chips, or other electronic components that can transmit and receive signals from other devices including the computing system 130 (e.g., the local server 140, the remote server 150, or both). For instance, the exercise equipment 120 may be configured or programmed to transmit the equipment attributes 124 and/or the equipment sensor data 122 to the computing system 130 (e.g., to the local server 140, the remote server 150, or both). The equipment transceiver 320c may be configured or programmed to communicate in accordance with any number of wired or wireless communication protocols. For instance, the communication transceiver may be programmed to communicate in accordance with a satellite-communication protocol, a cellular-based communication protocol (5G, LTE, 3G, etc.), Bluetooth®, Bluetooth® Low Energy, Ethernet, WiFi, ANT, etc.

The equipment memory 320d is implemented via circuits, chips, or other electronic components and can include one or more of read only memory (ROM), random access memory (RAM), flash memory, electrically programmable memory (EPROM), electrically programmable and erasable memory (EEPROM), embedded MultiMediaCard (eMMC), a hard drive, or any volatile or non-volatile media etc. The equipment memory 320d may store instructions executable by the equipment processor 320e and data such as the equipment attributes 124, the equipment sensor data 122, or the like. The instructions and data stored in the equipment memory 320d may be accessible to the equipment processor 320e and possibly other components of the exercise equipment 120, such as the equipment transceiver 320c.

The equipment processor 320e is implemented via circuits, chips, or other electronic components and may include one or more microcontrollers, one or more field programmable gate arrays (FPGAs), one or more application specific integrated circuits (ASICs), one or more digital signal processors (DSPs), one or more customer specific integrated circuits, etc. The equipment processor 320e is configured or programmed to process and/or transmit the equipment attributes 124 and/or the equipment sensor data 122. The equipment processor 320e may be further configured or programmed to command the machine sensors 320a to operate and collect the equipment sensor data 122 therefrom.

The equipment sensor data 122 may include motion information of the mechanical input 320a in raw form, such as distance traveled vs. time, or as processed from the raw data by the equipment processor 320e or the computing system 130. As processed from the raw data, the equipment sensor data 122 may quantify various characteristics of exercise performed with the exercise equipment 120, which may be referred to as equipment exercise characteristics that may be processed with the user sensor data 112, such as the user exercise characteristics, to associate the users with the exercise equipment 120 used thereby at given times. The equipment exercise characteristics may include, for example: repetition start, peak, and end distances and corresponding times; repetition travel and corresponding repetition number or time; performance of a repetition and time; repetition duration and corresponding repetition number or time; and number of repetitions in a set and/or set duration and corresponding time. The equipment setting or configuration may be detected and may influence the equipment attributes 124 for exercises being performed in a given configuration, such as the exercise performed (e.g., chest vs. shoulder press) and/or expected motion (e.g., repetition motion and/or orientation).

TABLE 3
Equipment Exercise Characteristics

| Motion Information | Description; Source |
|---|---|
| Distance traveled | Measured from motion sensor of equipment sensors 320b |
| Repetition start distance | Repeated position of mechanical input 320a at start of each |
| | repetition |
| Repetition peak distance | Repeated position of mechanical input 320a at the end of each |
| | repetition, generally furthest from the start position |
| Repetition end distance | Repeated position of mechanical input 320a at the end of each |
| | repetition, generally forming the start position of each |
| | successive repetition |
| Repetition travel | Distance between start and peak positions of mechanical input |
| | 320a |
| Performance of a repetition | Binary determination of whether and when a repetition was |
| | performed with the exercise equipment |
| Repetition duration | Time span between start and end positions |
| Number of repetitions | Number of times moving from starting to peak positions |
| Set duration | Time span between start of first repetition and end of last |
| | repetition |
| Equipment setting or | Selectable setting (e.g., resistance) or physical configuration of |
| configuration | exercise equipment 120 (e.g., arm position of a cable press |
| | machine, inclination of a treadmill) |

The processed motion information of the exercise equipment 120 outlined in Table 3, as measured by the equipment sensors 320b, may correspond to the user exercise characteristics outlined in Table 1 and be processed in accordance therewith (e.g., being compared thereto) in order to identify the user performing an exercise on particular exercise equipment 120 for which the equipment sensor data 122 is collected. For example, the repetition start, peak, and end positions, repetition distance, repetition duration, number of repetitions, and set duration from the user sensor data 112 of the user may correspond to the repetition start, peak, and end distances, repetition travel, repetition duration, number of repetitions, and set duration of the equipment sensor data 122 of the exercise equipment 120. Similarly, the repetition path, repetition start, peak, and end orientations, and repetition orientation path of the user sensor data 112 of the user may correspond to the equipment attributes 124 of the exercise equipment for a singular or different configurations, as outlined in Table 2 above. In another example, the identified exercise, timing, and other user exercise characteristic (e.g., cadence) identified for the user may correspond to the exercise of the equipment attributes 124 and the timing or other equipment exercise characteristic (e.g., cadence) of the exercise equipment 120.

FIG. 4 is a block diagram illustrating example components of the local server 140. As shown in FIG. 3, the local server 140 includes a local server transceiver 440a, a local server memory 440b, and a local server processor 440c.

The local server transceiver 440a is implemented via an antenna, circuits, chips, or other electronic components that facilitate wireless communication between the local server 140 and the wearable device 110 and remote server 150. The local server transceiver 440a may be programmed to communicate in accordance with any number of wired or wireless communication protocols. For instance, the local server transceiver 440a may be programmed to communicate in accordance with a satellite-communication protocol, a cellular-based communication protocol (5G, LTE, 3G, etc.), Bluetooth®, Bluetooth® Low Energy, Ethernet, WiFi, ANT, etc. For instance, the local server transceiver 440a may be configured or programmed to receive the user sensor data 112 from the wearable device 110, the equipment sensor data 122 from the exercise equipment 120, as well as the exercise recommendations 132 and the exercise information 134 from the remote server 150. Further, the local server transceiver 440a may be configured or programmed to transmit the other user information 114 to the wearable device 110 and/or the remote server 150. In some instances, the local server transceiver 440a may be configured or programmed to transmit the user sensor data 112 and/or the equipment sensor data 122 to the remote server 150.

The local server memory 440b is implemented via circuits, chips, or other electronic components and can include one or more of read only memory (ROM), random access memory (RAM), flash memory, electrically programmable memory (EPROM), electrically programmable and erasable memory (EEPROM), embedded MultiMediaCard (eMMC), a hard drive, or any volatile or non-volatile media etc. The local server memory 440b may store instructions executable by the local server 140 and data such as the equipment sensor data 122, exercise recommendations 132, exercise information 134, the other user information 114, including the user profile, the equipment sensor data 122, or the like. The instructions and data stored in the local server memory 440b may be accessible to the local server processor 440c and possibly other components of the local server 140, such as the local server transceiver 440a.

The local server processor 440c is implemented via circuits, chips, or other electronic components and may include one or more microcontrollers, one or more field programmable gate arrays (FPGAs), one or more application specific integrated circuits (ASICs), one or more digital signal processors (DSPs), one or more customer specific integrated circuits, etc. The local server processor 440c is configured or programmed to access and process the user sensor data 112 received from the wearable device 110 and/or the equipment sensor data 122 from the exercise equipment 120, as well as the exercise information 134 and exercise recommendations 132 received from the remote server 150. The local server processor 440c may be further configured or programmed to command the local server transceiver 440a to transmit the user sensor data 112 and/or the equipment sensor data 122 to the remote server 150. In some instances, the local server processor 440c may be programmed to update the other user information 114 based at least in part on the exercise recommendations 132 and the exercise information 134 received from the remote server 150. Further, the local server processor 440c may be programmed to command the local server transceiver 440a to transmit updated user information 114, such as the updated user profile, to the wearable device 110. Alternatively or in addition, the local server processor 440c may be configured or programmed to generate and command the local server transceiver 440a to transmit an email or other wireless communication including the updated user information 114. Alternatively or in addition, the local server processor 440c may be programmed to update a website, or more specifically, a database with information accessible to the user via a website, to reflect the updated user information.

FIG. 5 is a block diagram illustrating example components of the remote server 150. As shown in FIG. 5, the remote server 150 includes a remote server transceiver 550a, a remote server memory 550b, and a remote server processor 550c.

The remote server transceiver 550a is implemented via an antenna, circuits, chips, or other electronic components that facilitate wireless communication between the remote server 150 and the local server 140 and possibly the wearable device 110 and/or the exercise equipment 120. The remote server transceiver 550a may be programmed to communicate in accordance with any number of wired or wireless communication protocols. For instance, the remote server transceiver 550a may be programmed to communicate in accordance with a satellite-communication protocol, a cellular-based communication protocol (5G, LTE, 3G, etc.), Bluetooth®, Bluetooth® Low Energy, Ethernet, WiFi, ANT, etc. For instance, the remote server transceiver 550a may be configured or programmed to receive the user sensor data 112 and/or the equipment sensor data 122 from the local server 140, the wearable device 110, and/or the exercise equipment 120, as well as the other user information 114 from the local server 140 and the equipment attributes 55124 from exercise equipment 120 or other source. Moreover, in some possible approaches, the remote server transceiver 550a may be configured or programmed to transmit the exercise recommendations 132 and the exercise information 134 to the local server 140, the wearable device 110, or both.

The remote server memory 550b is implemented via circuits, chips, or other electronic components and can include one or more of read only memory (ROM), random access memory (RAM), flash memory, electrically programmable memory (EPROM), electrically programmable and erasable memory (EEPROM), embedded MultiMediaCard (eMMC), a hard drive, or any volatile or non-volatile media etc. The remote server memory 550b may store instructions executable by the remote server 150 and data such as the user sensor data 112 captured by the wearable device 110, the other user information 114 transmitted from the local server 140, the exercise recommendations 132 and the exercise information 134 generated by the remote server processor 550c, the equipment sensor data 122 and/or equipment attributes 124 received from the exercise equipment 120, or the like. The instructions and data stored in the remote server memory 550b may be accessible to the remote server processor 550c and possibly other components of the remote server 150, such as the remote server transceiver 550a.

The remote server processor 550c is implemented via circuits, chips, or other electronic components and may include one or more microcontrollers, one or more field programmable gate arrays (FPGAs), one or more application specific integrated circuits (ASICs), one or more digital signal processors (DSPs), one or more customer specific integrated circuits, etc. The remote server processor 550c is configured or programmed to access and process the user sensor data 112 received from the wearable device 110 or local server 140, the user information 114 received from the local server 140, and the equipment sensor data 122 and/or the equipment attributes 124 received from and/or associated with the exercise equipment 120. By processing the user sensor data 112, the user information 114, the equipment sensor data 122, and/or the equipment attributes 124, the remote server processor 550c can generate the exercise recommendation 132 and the exercise information 134. The remote server processor 550c may be further configured or programmed to command the remote server transceiver 550a to transmit the exercise recommendation 132 and the exercise information 134 to the local server 140, the wearable device 110, or both.

The remote server processor 550c may be configured or programmed to process the user sensor data 112, the equipment sensor data 122, the other user information 114, and the equipment attributes 124 using machine learning techniques or by otherwise comparing the user sensor data 112, the equipment sensor data 122, other user information 114, and equipment attributes 124 to each other and/or other information stored in one or more databases, for example, from previous iterations of generating the exercise recommendations 132 and the exercise information 134 for the same or different users. As a result of such processing, whether by whether by comparing and/or by using various machine-learning techniques, the remote server processor 550c may be configured or programmed to generate the exercise recommendation 132 and the exercise information 134. For instance, the remote server processor 550c may determine the type of exercise being performed by the user, the amount of weight used, the number of repetitions performed, the number of sets performed, the user's rate of perceived exertion, the time under tension, the amount of time the user spent at a particular piece of exercise equipment, distance traveled, work performed, calories burned, average and peak power output, etc., which may form the exercise information 134 as a summary of the exercise performed by the user.

In some possible implementations, the remote server processor 550c associates different users with the different exercise equipment 120 being used thereby at any given time, thereby allowing the exercise recommendations 132 and the exercise information 134 to be derived from the equipment sensor data 122 collected by the exercise equipment 120 during an exercise to be sent to the wearable device 110 of the user having performed the exercise. In one example, the remote server processor 550c cooperatively processes the user sensor data 112 from one or more wearable devices 110 and the equipment sensor data 122 from one or more pieces of the exercise equipment 120 using machine learning techniques or by other forms of processing (e.g., step-wise comparisons of different forms of the user sensor data 112 and the equipment sensor data 122). The processing may further incorporate the other user information 114, the equipment attributes 124, or both.

In one implementation, the remote server processor 550c uses machine learning. For example, the remote server processor 550c identifies one or multiple candidates (e.g., wearable devices 110 and/or users) for exercise recommendations 132 and the exercise information 134 derived from the equipment sensor data 122 collected from one of the multiple pieces of the exercise equipment 120. The remote server processor 550c may assign a confidence score to each candidate and select the candidate with the highest confidence score based on the processing as the exercise recommendation 132 and the exercise information 134 to transmit to the local server 140, the wearable device 110, or both.

Identifying each candidate and determining confidence scores therefore may include the remote server processor 550c considering multiple datapoints. For example, the remote server processor 550c may identify one or more potential user candidates and determine confidence scores therefore by processing multiple of types of the user sensor data 112 of one or more users from the wearable devices 110 associated therewith with one or more different types of the equipment sensor data 122 of one or more pieces of the exercise equipment 120 and/or to the equipment attributes 124 of the one or more pieces of the exercise equipment 120. The types of user sensor data 112 and the equipment sensor data 122 may be in raw or processed form, such as those types described with respect to Tables 1 and 3, respectively, collected generally concurrently. The equipment attributes 124 may include those described with respect to Table 2.

In one example, the remote server processor 550c may be configured or programmed to consider the user sensor data 112 including the user's heartrate, the orientation of the user's hands, and the location of the user within a gym. The user sensor data 112 may, for instance, indicate that the user's heart rate is elevated, that the user's hands are pronated and moving back-and-forth, and that the user is located in a rowing machine section of the gym. From this user sensor data 112, the remote server processor 550c may determine, with relatively high confidence, that the user is using a rower. The remote server processor 550c may generate the exercise information 134 to reflect that the user is using a rower and transmit the exercise information 134 to the local server 140 or wearable device 110.

In another example, the remote server processor 550 may process one more of performance, distance, or duration of a repetition and repetition orientation of the user sensor data 112 in conjunction with performance, distance, or duration of a repetition of the equipment sensor data 122 and repetition orientation of the equipment attributes 124.

Alternative to machine learning, the remote server processor 550c may identify the user using other algorithms, such as comparing different types of the user sensor data 112 with the equipment sensor data 122 and/or the equipment attributes in a stepwise manner. For example, the remote server processor 550c may first compare the user sensor data 112 with the equipment sensor data 122 to determine which of the wearable devices 110 and the exercise equipment 120 exercises are being used at a given time to perform any exercise (e.g., concurrent motion is detected). If more than one of the wearable devices 110 and/or more than one of the pieces of the exercise equipment 120 are identified being concurrently used, the remote server processor 550c may subsequently compare other forms of the user sensor data 112 from the wearable devices 110 identified in use, such as those described with respect to Table 1, with other forms of the equipment sensor data 122 from the exercise equipment 120 also identified in use, such as those described with respect to Table 3, and/or the equipment attributes 124 thereof, such as those described with respect to Table 2. Based on favorable comparisons (e.g., similarity), particular users (e.g., the wearable devices 110 and the user sensor data 112 thereof) may be associated with the exercise equipment 120 and the equipment sensor data 122 being used thereby at a given time.

In these manners manner, the users are associated with the exercise equipment 120 being used thereby, such that the exercise recommendations 132 and/or the exercise information 134 may be provided to the user (e.g., to the wearable device 110 or personal computing device 116 thereof) without any direct interaction or other association between the wearable device 110 or the user and the exercise equipment 120 on which the user performs an exercise. For example, the fitness system 100 may associate the user with the exercise equipment 120 being used thereby without a direct electronic exchange of information therebetween, without providing identifying information of the specific piece of the exercise equipment 120 to the wearable device 110 or the personal computing device 116 associated therewith, and without providing identifying information of the wearable device 110 or the user to the exercise equipment 120. This association without a direct input between the wearable device and the exercise equipment 120 may ease the use and receipt of the recommendations 132 and/or the exercise information 134 by the user. In other manners, however, such as with the wearable device sensor 210b including a visual sensor, the wearable device 110 may receive a direct input identifying the exercise equipment 120, such as by reading a QR code of the exercise equipment 120. While not associating the wearable device 110 with the exercise equipment 120, the reading of a QR code or other visual indicia may still significantly improve ease of use for the user as compared to manual input of the exercise information or identifying information of the exercise equipment 120.

In some instances, the remote server processor 550c may be configured or programmed to provide exercise recommendations 132, as discussed above. Using the foregoing example of the user at the rower, the remote server processor 550c may evaluate the other user information 114 such as information in the user profile, the user demographics, historical exercise information associated with the user, target exercise information, and user attributes to provide the exercise recommendations 132. For instance, the remote server processor 550c may determine, from the historical exercise information, that the user typically rows 2000 m in 9 minutes and 23 seconds with a resistance setting of 5 out of 10 and an average of 34 strokes per minute. The remote server processor 550c may determine, from the target exercise information, that the user wishes to increase their speed as measured in strokes per minute. The remote server processor 550c may generate the exercise recommendations 132 to suggest a target stroke count of 35 strokes per minute.

Other exercise recommendations 132 may include increasing an amount of weight lifted, increasing a time under tension, performing more repetitions, performing more sets, exercising auxiliary muscles, exercising for longer periods of time, exercising for shorter periods of time, stretching, etc.

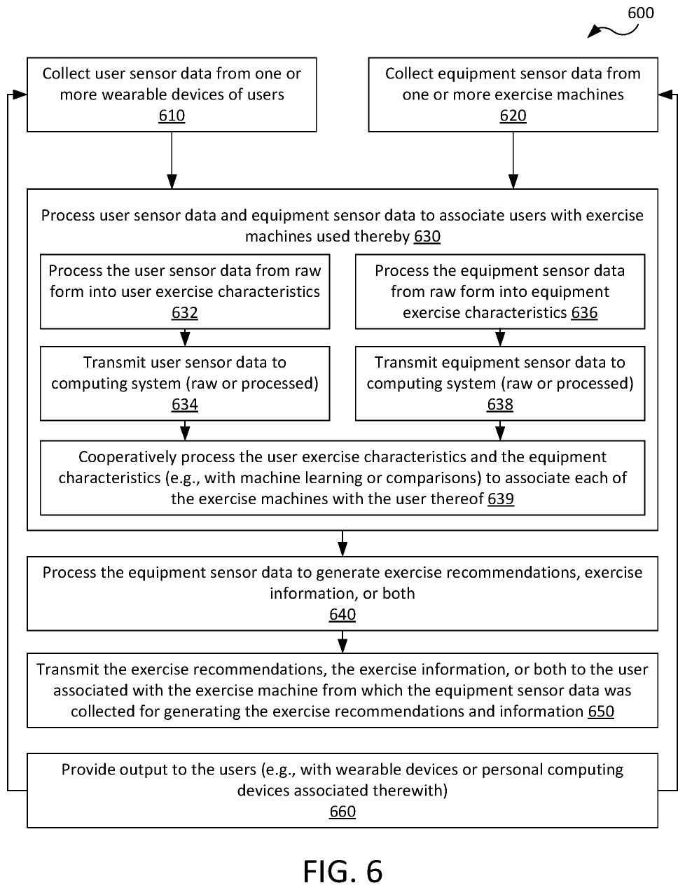

FIG. 6 is a flowchart of an example process 600 that may be executed by the fitness system 110. At block 610, the wearable devices 110 capture user sensor data 112 (e.g., with the sensors 210b thereof).

At block 620, which is performed simultaneous with block 610, the exercise equipment 120 captures equipment sensor data 122 (e.g., with the equipment sensors 320a thereof).

At block 630, the user sensor data 112 and the equipment sensor data 122 are processed to associate users (e.g., the wearable devices 110 thereof) the pieces of the exercise equipment 120 used thereby at different times.

Within block 630 at block 632, the user sensor data 112 any of the other user information 114 is transmitted to the computing system 130, such as from the wearable device 110 or the personal computing device 116 of the user. The user sensor data 112 and/or the other user information 114 may be sent in an anonymized manner for multiple users, for example, in conjunction with an anonymized user identifier for each of the users, as described previously.

Within block 630 at block 634, the user sensor data 112 (e.g., linear acceleration data, angular velocity data, or other data collected by an accelerometer, gyroscope, and/or IMU) may be processed by the wearable device 110 (e.g., the device processor 210e) or the computing system 130 (e.g., by a processor of a server thereof) to from raw form determine user exercise characteristics, as described above (e.g., performance of a repetition by the user, distance of a repetition, and/or number of repetitions performed be the user, exercise, cadence).

Block 632 and block 634 may be performed in any suitable order, for example, with the user sensor data 112 being processed by the wearable device 110 and subsequently transmitted to the computing system 130, or being transmitted by the wearable device 110 in raw form and subsequently processed by the computing system 130.

Within block 630 at block 636, the equipment sensor data 122 is transmitted to the computing system 130, such from the exercise equipment 120.

Within block 630 at block 638, the equipment sensor data 122 (e.g., motion data pertaining to the mechanical input 320a) may be processed by the exercise equipment 120 (e.g., the equipment processors 320e thereof) or the computing system 130 (e.g., by a processor of a server thereof) from raw form to determine equipment exercise characteristics, as described above (e.g., performance of a repetition, travel of a repetition, or number of repetitions performed with the exercise equipment 120).

Block 636 and block 638 may be performed in any suitable order, for example, with the equipment sensor data 122 being processed by the exercise equipment 120 and subsequently transmitted to the computing system 130, or being transmitted by the exercise equipment 120 in raw form and subsequently processed by the computing system 130.

Within block 630 at block 639, the user exercise characteristics of the user sensor data 112 and the equipment exercise characteristics of the equipment sensor data 122 are cooperatively processed by the computing system 130, such as described above with the remote server processor 550a of the remote server 550, to associate each of the different pieces of the exercise equipment 120 with the user thereof, such as with the wearable device 110 thereof. The cooperative processing of block 639 may include processing the user sensor data 112 and the equipment sensor data 122 with the other user information 114 and/or the equipment attributes 124. The cooperative processing may include filtering (e.g., associating en masse) those users at a particular exercise facility location having the exercise equipment (e.g., according to the other user information 114 or the user sensor data 112, and the equipment attributes 124), so as to limit the number of possible associations that require processing.

At block 640, the equipment sensor data 122 is processed, alone or in conjunction with the user sensor data 112, the other user information 114, to generate the exercise recommendations 132, the exercise information 134, or combinations thereof. Block 640 is performed by the computing system 130, such as by the remote server processor 550c of the remote server 150 as described previously.

At block 650, the exercise recommendations 132, the exercise information 134, or both, are transmitted to the user associated with the piece of exercise equipment 120 from which the equipment sensor data 122 was collected and used to generate the exercise recommendations 132 and/or the exercise information 134, such as to the wearable device 110 thereof. Block 650 is performed by the computing system 130, such as by the remote server transceiver 550a of the remote server.

At block 660, one or more of the outputs 118 are provided to the user, for example, by the wearable device 110 and/or the personal device 116 of the user according to the recommendations 132 and/or the exercise information 134. The output 118 may, for example, be an alert or an instruction, as described previously.

Blocks 610, 620, 630, 640, 650, and 660 are repeated.

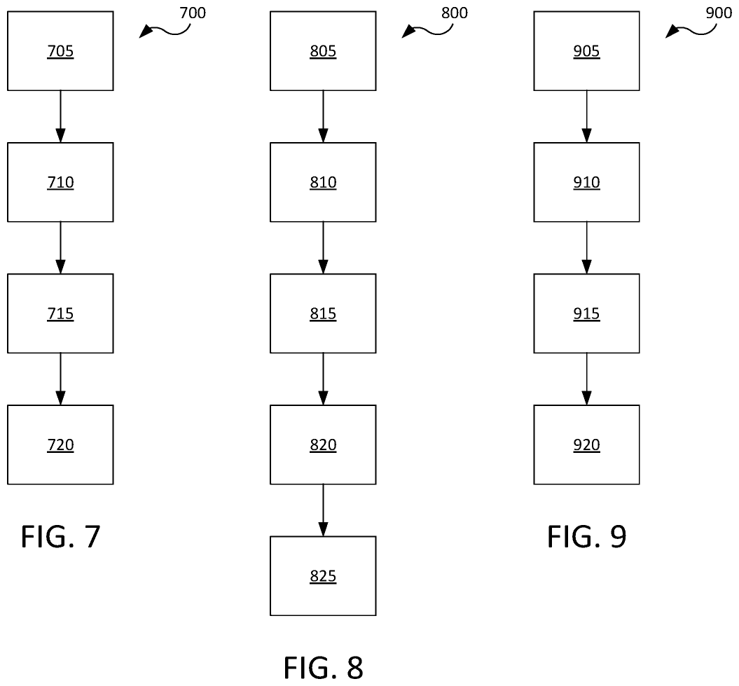

FIG. 7 is a flowchart of an example process 700 that may be executed by the wearable device 110. The process 700 may start at any time, such as when the user is wearing the wearable device 110 and may continue to execute until the user is no longer wearing the wearable device 110.

At block 705, the wearable device 110 captures user sensor data 112. For instance, the wearable device sensors 210b may capture biometric information 112a, motion information 112b, and location information 112c. The biometric information 112a may include the user's heart rate. The motion information 112b may represent the user's movements, hand positions, etc. (e.g., the linear acceleration and/or angular velocity data over time). The location information 112c may represent the relative or absolute location of the user. The wearable device sensors 210b may capture the user sensor data 112 and output the user sensor data 112 to the wearable device processor 210e.

At block 710, the wearable device 110 transmits the user sensor data 112 to the computing system 130 (e.g., local server 140, the remote server 150, or both). For instance, the wearable device processor 210e may be configured or programmed to command the wearable device transceiver 210c to transmit the user sensor data 112 to the local server 140, the remote server 150, or both.

At block 715, the wearable device 110 receives exercise recommendations 132 and the exercise information 134. For instance, the wearable device transceiver 210c receives communications from the computing system 130 (e.g., the local server 140, the remote server 150, or both), and the communications include the exercise recommendations 132 and the exercise information 134.

At block 720, the wearable device 110 provides a physical output to the user, such as an alert or an instruction, as described previously. For example, the wearable device 110 may present the exercise information 134 to the user. In one possible implementation, the wearable device processor 210e commands the physical output 210a to present the exercise recommendations 132 and the exercise information 134 to the user visually (e.g., with alphanumeric text).

FIG. 8 is a flowchart of an example process 800 that may be executed by the local server 140. The process 800 may run continuously over, e.g., a period of time user sensor data 112 is expected from the wearable device 110 and/or the equipment sensor data 122 is expected from the exercise equipment 120.

At block 805, the local server 140 receives user sensor data 112 from the wearable device 110. The local server transceiver 440a receives the user sensor data 112 transmitted from the wearable device 110 and/or the equipment sensor data 122 transmitted from the exercise equipment and passes the user sensor data 112 and/or the equipment sensor data 122 to the local server memory 440b, the local server processor 440c, or both.

At block 810, the local server 140 transmits the user sensor data 112, the equipment sensor data 122, and/or the other user information 114 to the remote server 150. For instance, the local server processor 440c may command the local server transceiver 440a to transmit the user sensor data 112, the equipment sensor data 122, and the other user information 114 to the remote server 150.

At block 815, the local server 140 receives exercise recommendations 132 and the exercise information 134 from the remote server 150. The local server transceiver 440a may receive signals from the remote server 150 that include the exercise recommendations 132 and the exercise information 134.

At block 820, the local server 140 transmits the exercise recommendations 132 and the exercise information 134 to the wearable device 110. For instance, the local server processor 440c may command the local server transceiver 440a to forward the exercise recommendations 132 and the exercise information 134 to the wearable device 110 associated with the user.

At block 825, the local server 140 updates the user profile to reflect the exercise recommendations 132 and the exercise information 134 received from the remote server 150. For instance, the local server processor 440c may be configured or programmed to update a database stored in the local server memory 440b with information contained in the exercise recommendations 132 and the exercise information 134.

FIG. 9 is a flowchart of an example process 900 that may be executed by the remote server 150. The process 900 may run continuously over, e.g., a period of time that the user sensor data 112 is collected by the wearable device 110 and/or the equipment sensor data 122 is collected by the exercise equipment 120 and transmitted to the remote server 150 from the local server 140.

At block 905, the remote server 150 receives the user sensor data 112, other user information 114, the equipment sensor data 122, and equipment attributes 124. The remote server transceiver 550a may receive the user sensor data 112 directly from the wearable device 110 or from the local server 140 and may receive the equipment sensor data 122 directly from the exercise equipment 120 or from the local server 140. The remote server transceiver 550a may receive the other user information 114 from the local server 140. The remote server transceiver 550a may receive the equipment attributes 124 from the exercise equipment 120.

At block 910, the remote server 150 processes the user sensor data 112, the other user information 114, the equipment sensor data 122, and the equipment attributes 124 to develop exercise recommendations 132 and the exercise information 134. For example, the remote server processor 550c may compare the user sensor data 112, the equipment sensor data 122, other user information 114, and equipment attributes 124 to each other and/or information stored in one or more databases stored in the remote server memory 550b. As a result of such comparison, and/or by using various machine-learning techniques, the remote server processor 550c may be configured or programmed to generate the exercise recommendation 132 and the exercise information 134. For instance, the remote server processor 550c may determine the type of exercise being performed by the user, the amount of weight used, the number of repetitions performed, the number of sets performed, the user's rate of perceived exertion, the time under tension, the amount of time the user spent at a particular piece of exercise equipment, etc. Moreover, generating the exercise recommendations 132 and the exercise information 134 may include identifying multiple candidates for exercise recommendations 132 and the exercise information 134, assign a confidence score to each candidate, and select the candidate with the highest confidence score as the exercise recommendation 132 and the exercise information 134. Further, as discussed above, the remote server processor 550c may compare multiple datapoints when developing the exercise recommendations 132 and the exercise information 134.

At block 915, the remote server 150 transmits the exercise recommendations 132 and the exercise information 134 to the local server 140, wearable device 110, or both. That is, the remote server processor 550c may command the remote server transceiver 550a to transmit the exercise recommendations 132 and the exercise information 134 to the local server 140 so the user profile may be updated. The remote server processor 550c may command the remote server transceiver 550a to transmit the exercise recommendations 132 and the exercise information 134 to the wearable device 110 so the exercise recommendations 132 and the exercise information 134 may be presented to the user. In some possible approaches, the remote server processor 550c commands the remote server transceiver 550a to transmit the exercise recommendations 132 and the exercise information 134 to the local server 140 so the local server 140 may transmit the exercise recommendations 132 and the exercise information 134 to the wearable device 110.

At block 920, the remote server 150 updates databases or tables stored in the remote server memory 550b to improve future exercise recommendations 132 and the exercise information 134. As discussed above, the remote server processor 550c may apply machine learning to improve the accuracy of the exercise recommendations 132 over time. The databases of the remote server memory 550b are updated so the remote server 150 can continuously learn and apply the most recent data when generating exercise recommendations 132 and the exercise information 134.

It should be noted that each of the blocks in the processes described above may be performed by any other suitable devices or systems (e.g., processing steps may be performed by multiple and/or different computing devices than described above).

FIG. 10 is a schematic diagram illustrating an example automatic activity detection and data pull performed by the fitness system 110. As illustrated, the wearable device 110 collects information including the time, number of repetitions performed, the location, a data feed from the exercise equipment (RSS), an exercise, a user heart rate, and information about the user. A piece of the exercise equipment 120 collects information such as the amount of work performed, the power output of the user, an amount of force exerted during the exercise, a weight setting, an exercise time, the number of repetitions performed, the location, a machine identifier, and a list of exercises available via the machine 120. The information from the wearable device 110 and the exercise equipment 120 are uploaded to the local server 140 or the remote server 150, either of which may generate a comprehensive data set from the user sensor data 112 captured by the wearable device 110 and the equipment sensor data 122 captured by the exercise equipment 120. The comprehensive data set may further include the user profile data. In some instances, the comprehensive data set may be generated as a result of comparing common data attributes, such as by comparing or otherwise processing the user exercise characteristics of the user sensor data 112 with the equipment exercise characteristics of the equipment sensor data 122 or the equipment attributes 124. At least some of the information transmitted from the wearable device 110 and the exercise equipment 120, such as the comprehensive data set, along with user profile data if not otherwise included in the comprehensive data set, can be transmitted to the remote server 150, which as discussed above, can generate exercise recommendations 132 and the exercise information 134 from the comprehensive data set, user profile, or both.

A first aspect of the disclosure relates to a server having a memory and a processor programmed to execute instructions stored in the memory. The instructions include receiving real-time data captured by a wearable device and generating an exercise recommendation based at least in part on the real-time data captured by the wearable device.

A second aspect of the disclosure relates to a method includes receiving, at a server, real-time data captured by a wearable device and generating an exercise recommendation based at least in part on the real-time data captured by the wearable device.

A third aspect of the disclosure relates to a method includes receiving, at a server, real-time data captured by a piece of exercise equipment, receiving, at a server, real-time data captured by a wearable device, and creating a comprehensive data set including the data received from the exercise equipment and the wearable device. In the third approach, creating the comprehensive data set includes comparing common data attributes.

A fourth aspect of the disclosure relates to a method according to the third aspect further including transmitting at least some of the comprehensive data set to the wearable device.

A fifth aspect of the disclosure relates to a method according to the fourth aspect further including generating exercise recommendations based at least in part on information contained in the comprehensive data set and transmitting the exercise recommendations to the wearable device.

A sixth aspect of the disclosure relates to a method according to the fifth aspect wherein generating the exercise recommendations includes generating the exercise recommendations based at least in part on user profile information stored in a server.

A seventh aspect of the disclosure relates to receiving, at a server, real-time data captured by a piece of exercise equipment, receiving, at a server, real-time data captured by a wearable device, comparing common data attributes of the real-time data captured by the piece of exercise equipment and the real-time data captured by the wearable device to create a comprehensive data set, and generating an exercise recommendation based at least in part on the real-time data captured by the exercise equipment.

An eighth aspect of the disclosure relates to a server having a memory and a processor programmed to execute instructions stored in the memory, the instructions including receiving real-time data captured by a wearable device, the real-time data including a first data point and a second data point and generating an exercise recommendation based at least in part on the first data point and the second data point captured by the wearable device.

In general, the computing systems and/or devices described may employ any of a number of computer operating systems, including, but by no means limited to, versions and/or varieties of the Microsoft Windows® operating system, the Unix operating system (e.g., the Solaris® operating system distributed by Oracle Corporation of Redwood Shores, California), the AIX UNIX operating system distributed by International Business Machines of Armonk, New York, the Linux operating system, the OS X, macOS, and iOS operating systems distributed by Apple Inc. of Cupertino, California, the BlackBerry OS operating system distributed by Blackberry, Ltd. of Waterloo, Canada, and the Android operating system developed by Google, Inc. and the Open Handset Alliance. Examples of computing devices include, without limitation, a computer workstation, a server, a desktop, notebook, laptop, or handheld computer, or some other computing system and/or device.

Computing devices generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. Some of these applications may be compiled and executed on a virtual machine, such as the Java Virtual Machine, the Dalvik virtual machine, or the like. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A fitness system comprising:
multiple pieces of exercise equipment configured to be operated by users to perform different physical exercises therewith, each of the multiple pieces of exercise equipment including an equipment sensor that senses equipment motion and outputs equipment sensor data according thereto;
wearable devices, each associated with a different one of the users and including a user sensor that senses user motion that outputs user sensor data according thereto; and
a computing system that cooperatively processes the equipment sensor data from the multiple pieces of exercise equipment and the user sensor data from the wearable devices to associate each of the users with a different one of the multiple pieces of exercise equipment being used thereby and, for each user, generate an exercise recommendation from the equipment sensor data from the one piece of exercise equipment being used thereby;
wherein for each user, the computing system transmits to the wearable device of the user the exercise recommendation generated therefor; and
wherein each wearable device provides a physical output according to the exercise recommendation transmitted thereto.

2. A fitness system comprising:
a computing system in communication with a wearable device and multiple pieces of exercise equipment, wherein the wearable device is associated with a user, includes a user sensor for detecting motion of the user, and outputs user sensor data according to the detecting of the motion of the user, and wherein each one of the multiple pieces of exercise equipment is configured to be used by the user to perform an exercise therewith, includes an equipment sensor for detecting motion thereof, and outputs equipment sensor data according to the detecting of the motion thereof;
wherein the computing system:
receives the user sensor data from the wearable device and the equipment sensor data from the multiple pieces of exercise equipment;
processes the user sensor data collected at a first time and the equipment sensor data collected from the multiple pieces of exercise equipment at the first time to associate the user with one of the multiple pieces of exercise equipment used by the user at the first time;
determines exercise information, exercise recommendations, or both from the equipment sensor data collected from the one piece of exercise equipment at the first time; and
transmits the exercise information, the exercise recommendations, or both.

3. The fitness system according to claim 2, wherein the wearable device provides a physical output according to the exercise information, the exercise recommendations, or both, the physical output including one or more of a visual output, an audible output, or a tactile output.

4. The fitness system according to claim 3, wherein the physical output includes an instruction according to the exercise information directing the user to perform an action pertaining to the exercise currently being performed by the user.

5. The fitness system according to claim 3, wherein the physical output includes an alert regarding the exercise recommendation, the exercise information, or both having been received by the wearable device.

6. The fitness system according to claim 2, wherein the computing system is in communication with another wearable device associated with another user, and the other wearable device includes another user sensor for detecting motion of the other user, and outputs other user sensor data according to the detecting the motion of the other user;
wherein the computing system:
receives the other user sensor data from the other wearable devices;
processes the other user sensor data collected at the first time and the equipment sensor data collected at the first time to associate the other user with another piece of the multiple pieces of exercise equipment being used by the other user at the first time;
determines other exercise information, other exercise recommendations, or both for the other user from the other equipment sensor data collected from the other piece of exercise equipment at the first time; and
transmits the other exercise information, the other exercise recommendations, or both.

7. The fitness system according to claim 6, wherein the computing system processes the user sensor data collected at a second time and the equipment sensor data collected from the multiple pieces of exercise equipment at the second time to associate the user with another one of the multiple pieces of exercise equipment being used by the user at the second time;
determines second exercise information, second exercise recommendations, or both from the equipment sensor data collected from the other piece of exercise equipment at the second time; and
transmits the second exercise information, the second exercise recommendations, or both.

8. The fitness system according to claim 6, wherein the computing system processes the user sensor data, the other user sensor data, and the equipment sensor data with machine learning to the user with the one piece of exercise equipment at the first time and the other user with the other piece of exercise equipment at the second time.

9. The fitness system according to claim 2, wherein the computing system determines a user exercise being performed by the user from the user sensor data collected at the first time, and compares the exercise to equipment attributes of the multiple pieces of exercise equipment to associate the user with the one piece of exercise equipment used by the user at the first time, the equipment attributes including one or more equipment exercises that may be performed by the exercise equipment.

10. The fitness system according to claim 2, wherein the user sensor data includes at least one of linear acceleration data or angular velocity data.

11. The fitness system according to claim 10, wherein one of the wearable device or the computing system determines from the user sensor data for the first time a user exercise characteristic, the user exercise characteristic including at least one of performance of a repetition by the user, distance of a repetition performed by the user, or a number of repetitions performed by the user;
wherein one of the multiple pieces of exercise equipment or the computing system determines from the equipment sensor data for the first time an equipment exercise characteristic, the equipment exercise characteristic including at least one of performance, distance, or number of repetitions performed with the one piece of exercise equipment; and
wherein the computing system processes the user sensor data and the equipment sensor data by comparing the user exercise characteristic of the first time with the equipment exercise characteristic of the first time to associate the user with the one piece of exercise equipment used by the user at the first time.

12. The fitness system according to claim 2, wherein the wearable device and the multiple pieces of exercise equipment do not receive inputs associated with each other independent of the computing system.

13. The fitness system according to claim 2, wherein the computing system determines an exercise recommendation from the equipment sensor data collected from the one piece of exercise equipment at the first time and transmits the exercise recommendation to the wearable device associated with the one piece of exercise equipment at the first time, the exercise recommendation including information about another exercise for the user to perform at a future time.

14. The fitness system according to claim 2, further comprising the wearable device.

15. The fitness system according to claim 2, further comprising the multiple pieces of exercise equipment.

16. The fitness system according to claim 2, comprising the wearable device and the computing system;
wherein the computing system determines a user exercise being performed by the user from the user sensor data collected at the first time, and compares the exercise to equipment attributes of the multiple pieces of exercise equipment to associate the user with the one piece of exercise equipment used by the user at the first time, the equipment attributes for each of the multiple pieces of the exercise equipment including one or more equipment exercises that may be performed thereby;
wherein the wearable device provides a physical output according to the exercise information, the exercise recommendations, or both, the physical output including one or more of a visual output, an audible output, or a tactile output, and wherein the physical output includes an instruction according to the exercise information directing the user to perform an action pertaining to the exercise currently being performed by the user; and
wherein the wearable device and the multiple pieces of exercise equipment do not receive inputs associated with each other independent of the computing system.

17. A fitness system comprising:
a computing system that:
receives user sensor data collected with multiple wearable devices individually associated with different users and equipment sensor data collected with exercise equipment, the exercise equipment including multiple pieces of the exercise equipment; and
cooperatively processes the user sensor data and the equipment sensor data to associate each of the wearable devices at different times with one piece of the exercise equipment being used at the different times by each of the users associated with each of the wearable devices.

18. The fitness system according to claim 17, wherein the user sensor data is collected by user motion sensors of the wearable devices, and the equipment sensor data is collected by equipment motion sensors of the exercise equipment;
wherein the computing system cooperatively processes equipment attributes with the user sensor data and the equipment sensor data to associate each of the wearable devices with the one piece of the exercise equipment, the equipment attributes being predefined characteristics of exercises performed with the exercise equipment; and
wherein the computing system further processes the equipment sensor data to produce exercise information for the different times, and transmits the exercise information to the wearable devices associated with the exercise equipment at the different times.

19. The fitness system according to claim 17, wherein the user sensor data is collected by user motion sensors of the wearable devices, and the equipment sensor data is collected by equipment motion sensors of the exercise equipment.

20. The fitness system according to claim 17, wherein the computing system cooperatively processes equipment attributes with the user sensor data and the equipment sensor data to associate each of the wearable devices at the different times with the one piece of the exercise equipment, the equipment attributes being predefined characteristics of exercises performed with the exercise equipment.

21. The fitness system according to claim 17, wherein the computing system further processes the equipment sensor data to produce exercise information for the different times, and transmits the exercise information to the wearable devices associated with the exercise equipment at the different times.
